(19) United States
(12) Patent Application Publication
(10) Pub. No.: US 2024/0173295 A1
(43) Pub. Date: May 30, 2024

(54) A STABLE, EMULSION-FORMING LIQUID COMPOSITION COMPRISING AMINO ACIDS

(71) Applicant: Brain Luxury Inc., Brooklyn, NY (US)

(72) Inventors: Axel Bouchon, Kleinmachnow (DE); Herman Schützinger, Salzburg (AT)

(21) Appl. No.: 18/552,064

(22) PCT Filed: Mar. 25, 2022
(86) PCT No.: PCT/US2022/022026

§ 371 Date: Sep. 22, 2023

Related U.S. Application Data
(60) Provisional application No. 63/165,885, filed on Mar. 25, 2021.

(51) Int. Cl. A61K 31/405; A61K 9/00; A61K 9/107; A61K 31/198; A61K 31/355; A61K 31/593; A61K 47/12; A61K 47/22; A61K 47/46; A61P 25/24

(52) U.S. Cl. CPC A61K 31/405; A61K 9/0053; A61K 9/0095; A61K 9/107; A61K 31/198; A61K 31/355; A61K 31/593; A61K 47/12; A61K 47/22; A61K 47/46; A61P 25/24

(57) ABSTRACT

The disclosure is in part directed to shelf-stable, emulsion-forming liquid compositions, liquid beverage compositions, and fatty acid vesicle-forming liquid compositions for increased neurosystem uptake of amino acids present in the compositions when consumed by a subject.

### BACKGROUND

Proteins commonly found in foods do not necessarily provide an amino acid composition that provides the amino acid requirements or needs of a mammal, such as a human, in an efficient manner. For example, it can be difficult to attain a meaningful threshold of certain amino acids in the brain. The brain is known to be acutely sensitive to changes in plasma amino acid concentrations within the physiologic range. An example of this sensitivity is the inhibition in cerebral protein synthesis caused by hyperphenylalaninemia and the reversal of this inhibition by the co-administration of other large neutral amino acids that compete with phenylalanine for transport into the brain from blood.

Further, certain amino acids play key roles in the nervous system (e.g., the central nervous system), for example, by serving as biosynthetic precursors for neurotransmitters or by serving as neurotransmitters themselves. For example, tryptophan may affect serotonin function, while tyrosine and/or phenylalanine may affect dopamine function. Typically, however, these amino acids often do not have quick availability to the brain and other neurosystems and therefore can be difficult to use, e.g., to provide assistance for healthier sleep and mood. For example, tryptophan, tyrosine, and phenylalanine each require a carrier protein for transport across the blood-brain barrier. If the amount of tryptophan entering the brain is limited, the amount of serotonin and melatonin being synthesized in the brain is also limited. Such reduced levels of serotonin in the brain are often manifested, for example, by alterations in sleep, mood, and overall well-being, as well as being associated with depression.

Mixtures comprising essential amino acids (e.g., tryptophan) have been used that hydrolyze a protein with relatively high levels of essential amino acids, such as whey protein, and/or by combining free amino acids in a mixture that optionally also includes a hydrolyzed protein such as whey. However, mixtures of this type may have a bitter taste, undesirable mouthfeel, the potential to create gastrointestinal upset and are poorly soluble, and may be deemed unsuitable or undesirable for certain uses.

Accordingly, it would be useful in certain situations to have compositions for supplying proteins that may e.g., facilitate amino acid delivery upon mammalian consumption, and may e.g. support natural sleep and that are also substantially shelf stable and palatable.

### SUMMARY

The present disclosure is directed, in one embodiment, to an emulsion-forming liquid composition, comprising: a) an aqueous liquid composition having a pH of about 3.0 to about 3.9 as measured at 20° C., comprising at least two different amino acids each independently selected from the group consisting of tryptophan, 5-hydroxytryptophan, glycine, tyrosine, and phenylalanine; and b) a fatty acid composition comprising at least one omega-9 fatty acid, at least one omega-6 fatty acid; and at least three omega-3 fatty acids; wherein the aqueous liquid composition and the fatty acid composition form an amino acid/fatty acid buffer system allowing the creation of a fatty acid vesicle-based emulsion when the aqueous liquid composition and the fatty acid composition are shaken together.

For example, disclosed herein is an emulsion-forming liquid composition, comprising: a) an aqueous liquid composition having a pH of about 3.0 to about 3.9 as measured at 20° C., comprising at least two different amino acids each independently selected from the group consisting of tryptophan, 5-hydroxytryptophan, glycine, tyrosine, and phenylalanine; and b) a fatty acid composition comprising at least three omega-3 fatty acids; wherein the aqueous liquid composition and the fatty acid composition form an amino acid/fatty acid buffer system allowing the creation of a fatty acid vesicle-based emulsion when the aqueous liquid composition and the fatty acid composition are shaken together.

Also provided herein is a liquid beverage composition comprising L-tryptophan; glycine; one or more omega-6 fatty acids; one or more omega-9 fatty acids; and three or more omega-3 fatty acids, wherein the weight ratio of omega-6 fatty acids: omega-9 fatty acids: omega-3 fatty acids is about 1: about 1.7 to about 2.3: about 5.7 to about 6.3; and an effective amount of a food-compatible acidic agent, wherein the liquid beverage composition has a pH of about 3.2 to about 3.6 as measured at 20° C.

Further disclosed herein is a liquid beverage composition comprising: flaxseed oil, algae oil, a plant-derived oil, L-tryptophan, and glycine; wherein the omega-6 fatty acids, omega-9 fatty acids and omega-3 fatty acids present in the flaxseed oil, algae oil, and plant-derived oil combined are present in a weight ratio of omega 6 fatty acids: omega 9 fatty acids: omega 3 fatty acids of about 1: about 1.7 to about 2.3: about 5.7 to about 6.3.

Additionally, disclosed herein is a liquid beverage composition comprising: flaxseed oil, algae oil, a plant-derived oil. L-tryptophan, and glycine; wherein the eicosapentaenoic acid, docosahexaenoic acid, and α-linoleic acid present in the flaxseed oil, plant-derived oil, and algae oil combined are present in a weight ratio of eicosapentaenoic acid: docosahexaenoic acid: α-linoleic acid of about 1: about 1.8 to about 2.2: about 3.5 to about 4.1.

Also disclosed herein is a fatty acid vesicle-forming liquid composition comprising, per 60 ml of the liquid composition: about 1-5 ml flaxseed oil; about 1-5 ml algae oil, about 1-5 ml of a plant-derived oil (wherein the plant-derived oil is selected from the group consisting of olive oil, avocado oil, coconut oil, and mixtures thereof): about 2000 to about 5000 mg glycine: about 350 to about 700 mg L-tryptophan; and about 47 to about 57 ml of a food-compatible aqueous solution with a pH of about 3.0 to about 3.9 at 20° C.; wherein when the liquid composition is shaken for 10 seconds or longer, the creation of fatty acid vesicles occurs.

For example, disclosed herein is a fatty acid vesicle-forming liquid composition comprising, per 60 ml of the liquid composition: about 2 ml flaxseed oil; about 2 ml algae oil; about 2 ml of a plant-derived oil (wherein the plant-derived oil is selected from the group consisting of olive oil, avocado oil, coconut oil, and mixtures thereof): about 3000 mg glycine; about 450 mg L-tryptophan; and about 54 ml of a food-compatible aqueous solution with a pH of about 3.0 to about 3.9 at 20° C.: wherein when the liquid composition is shaken for 10 seconds or longer, the creation of fatty acid vesicles occurs.

Also provided herein, for example, is method of improving sleep, well-being and/or mood in a human subject in need thereof, comprising administering to the human subject an effective amount of a composition disclosed herein, for example, a disclosed emulsion-forming liquid composition, a disclosed liquid beverage composition, or a disclosed fatty acid vesicle-forming liquid composition.

Further disclosed herein, for example, is a method of treatment or prophylaxis of depression in a human subject in need thereof, comprising administering to the human subject an effective amount of a composition disclosed herein, for example, a disclosed, emulsion-forming liquid composition, a disclosed liquid beverage composition, or a disclosed fatty acid vesicle-forming liquid composition.

### BRIEF DESCRIPTION OF THE DRAWINGS

FIG. 1A depicts a group analysis of self-assessed feelings of morning happiness in a study population before and after consumption of a disclosed liquid composition. FIG. 1B depicts a group analysis of self-assessed depressed mood in a study population of 22 subjects before and after consumption of a disclosed liquid composition.

FIG. 2A. FIG. 2B and FIG. 2C depict a group analysis for self-assessed insomnia and sleep issues in various periods of the night in a study population before and after consumption of a disclosed liquid composition.

FIG. 3A depicts a group analysis of self-assessed sleep intensity in a study population before and after consumption of a disclosed liquid composition. FIG. 3B depicts a group analysis of self-assessed sleep quality in a study population before and after consumption of a disclosed liquid composition.

FIG. 4A depicts a group analysis of self-assessed work and activities next day indicating general energy level in a study population before and after consumption of a disclosed liquid composition. FIG. 4B depicts a group analysis of anxiety in a study population before and after consumption of a disclosed liquid composition.

FIG. 5A and FIG. 5B depict a group analysis for self-assessed feelings of morning happiness in a study population before and after consumption of a disclosed liquid composition.

FIG. 6A and FIG. 6B depict a group analysis for self-assessed sleep quality in a study population before and after consumption of a disclosed liquid composition.

FIG. 7A and FIG. 7B depict a group analysis for self-assessed time needed to fall asleep in a study population before and after consumption of a disclosed liquid composition.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a±10% variation from the nominal value unless otherwise indicated or inferred. All ranges recited herein include the endpoints, including those that recite a range “between” two values.

“Treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness.” unless otherwise indicated.

The term “effective amount” means the amount of a disclosed liquid composition required to achieve a desired biological effect in a human cell, tissue, organ, or system. The term “subject” refers to a human subject.

As used herein, a “shelf-stable” liquid composition refers to a disclosed liquid composition that has been processed or packaged in order to withstand prolonged storage.

As used herein, the terms “amphiphilic amino acids” and “amphipathic amino acids” are used interchangeably and refer to those amino acids having both polar and nonpolar characteristics, e.g., those amino acids having both hydrophilic and hydrophobic properties.

As used herein, the term “plant-derived oil” refers to an oil derived from a plant source as opposed to an animal source or a petroleum source.

### Compositions

Disclosed herein in an embodiment is an emulsion-forming liquid composition, that may, for example, increase neurosystem uptake of certain amino acids present in the disclosed emulsion-forming liquid composition when consumed in emulsion form by a subject, comprising: a) an aqueous liquid composition having a pH of about 3.0 to about 3.9 at 20° C., where the aqueous liquid composition comprises at least two amino acids each independently selected from the group consisting of tryptophan, 5-hydroxytryptophan, glycine, tyrosine, and phenylalanine; and b) a fatty acid composition comprising at least one omega-9 fatty acid, at least one omega-6 fatty acid; and at least three omega-3 fatty acids. The aqueous liquid composition and the fatty acid composition, when shaken together, form an amino acid/fatty acid buffer system allowing the creation of a fatty acid vesicle-based emulsion. Contemplated herein is a composition that where the aqueous liquid composition and the fatty acid composition are contained within one container. In other embodiments, the aqueous liquid composition and the fatty acid composition may be contained within separate containers and then combined before the emulsion-forming liquid composition is shaken.

The aqueous liquid composition portion of the disclosed liquid composition may be a fruit juice. A contemplated fruit juice may be, but not limited to, apple juice, apricot juice, blackberry juice, black current juice, blueberry juice, boysenberry juice, calamansi juice, cantaloupe juice, cherry juice, elementine juice, cranberry juice, gooseberry juice, grape juice, grapefruit juice, guava juice, honeydew juice, lime juice, lime juice such as kaffir lime juice and/or key lime juice, kiwifruit juice, kumquat juice, lemon juice, lingonberry juice, lychee juice, mandarin orange juice, mango juice, Marion berry juice, mulberry juice, muskmelon juice, nectarine juice, orange juice, papaya juice, passion fruit juice, peach juice, pear juice, persimmon juice, pineapple juice, plum juice, pomegranate juice, prune juice, raspberry juice, red currant juice, strawberry juice, tangerine juice, watermelon juice, winter melon juice, and/or combinations thereof. In some embodiments, the fruit juice comprises lemon juice. In other embodiments, the fruit juice comprises lime juice. In further embodiments, the fruit juice comprises lemon juice and lime juice. Contemplated aqueous liquid compositions that form part of a disclosed composition may further include water, coconut water, flavored mineral water and/or other flavored water.

Contemplated aqueous liquid composition portions of the disclosed liquid composition may include two or more different amino acids each of which may independently be, but are not limited to, e.g., alanine, arginine, asparagine, aspartic acid, cysteine, glutamic acid, glutamine, glycine, histidine, isoleucine, lysine, methionine, phenylalanine, proline, selenocysteine, serine, threonine, tryptophan, 5-hydroxytryptophan, tyrosine, and valine. In other embodiments, the aqueous liquid composition comprises, for example, two or more essential amino acids each of which may independently be, but are not limited to, e.g., histidine, isoleucine, leucine, lysine, methionine, phenylalanine, threonine, tryptophan, and valine. In other embodiments, the aqueous liquid composition comprises, for example, two or more conditionally essential amino acids each of which may independently be, but are not limited to, e.g., arginine, cysteine, glycine, glutamine, proline, and tyrosine. In other embodiments, the aqueous liquid composition comprises, for example, two or more non-essential amino acids each of which may independently be, but are not limited to, e.g., alanine, aspartic acid, asparagine, glutamic acid, serine, and selenocysteine. In other embodiments, the aqueous liquid composition comprises, for example, two or more amphiphilic amino acids each of which may independently be, but are not limited to, e.g., glycine, lysine, phenylalanine, tryptophan, 5-hydroxytryptophan, and tyrosine. In other embodiments, the aqueous liquid composition comprises two or more different amino acids each independently selected from the group consisting of tryptophan, 5-hydroxytryptophan, glycine, tyrosine, and phenylalanine. In other embodiments, the aqueous liquid composition comprises two or more different amino acids each independently selected from the group consisting of tryptophan, glycine, tyrosine, and phenylalanine. In other embodiments, the aqueous liquid composition comprises two or more different amino acids each independently selected from the group consisting of tryptophan, 5-hydroxytryptophan, and glycine.

Amino acids present in the aqueous liquid composition portion of a disclosed liquid composition may each independently be an L-isomer or a mixture of D- and L-isomers. For example, the aqueous liquid composition may include L-tryptophan and glycine. In some embodiments, the L-tryptophan and glycine are present in a weight ratio of about 1: about 6 to about 7.

A contemplated emulsion-forming liquid composition may further include magnesium, zinc, and/or a salt or hydrate thereof. For example, in some embodiments the emulsion-forming liquid composition further comprises magnesium (e.g., magnesium L-ascorbate dihydrate) and zinc (e.g., zinc citrate trihydrate.)

The fatty acid composition component may include a weight ratio of omega-6 fatty acids: omega 9-fatty acids: omega-3 fatty acids of about 1: about 1.7 to about 2.3: about 5.7 to about 6.3, and/or provide a weight ratio of eicosapentaenoic acid: docosahexaenoic acid: α-linoleic acid is about 1: about 1.8 to about 2.2: about 3.5 to about 4.1. Such fatty acid compositions may include one or more of, for example, flaxseed oil, algae oil, fish oil, and/or at least one plant-derived oil (e.g., coconut oil, avocado oil, and/or olive oil). Exemplary algae oils may be derived from algae found in marine or freshwater habitats. Exemplary algae oils may be derived from microalgae or macroalgae. Exemplary algae oils may include, for example, one or more of the following: brown algae oil, green algae oil, red algae oil, seaweed oil, kelp oil, rockweed oil, sargassum oil, Irish moss oil, coralline oil, dulse oil, sea lettuce oil, spirulina oil, chlorella oil, Schizochytrium sp. oil, and mixtures thereof. Exemplary plant-derived or plant-based oils may be derived from plants found in marine, freshwater or land habitats. Exemplary plant-derived or plant-based oils may include, for example, one or more of the following: almond oil, avocado oil, blackcurrant seed oil, brazil nut oil, canola oil, cashew oil, chia seed oil, coconut oil, corn oil, cottonseed oil, grapeseed oil, hazelnut oil, hemp seed oil, olive oil, palm oil, peanut oil, pecan oil, macadamia nut oil, perilla oil, pumpkin seed oil, rapeseed oil, safflower oil, seagrass oil, sea fennel oil, sesame oil, soybean oil, sunflower oil, walnut oil, and mixtures thereof. In some embodiments, the plant-derived oil is selected from the group consisting of coconut oil, avocado oil, olive oil, and mixtures thereof. In other embodiments, a plant-derived oil is coconut oil. In other embodiments, the plant-derived oil is avocado oil. In other embodiments, the plant-derived oil is olive oil. In other embodiments, a plant-derived oil is a mixture of olive oil and avocado oil. In other embodiments, a plant-derived oil is a mixture of olive oil and coconut oil. In other embodiments, a plant-derived oil is a mixture of avocado oil and coconut oil. In other embodiments, a plant-derived oil is a mixture of olive oil, avocado oil and coconut oil.

For example, a contemplated fatty acid composition that forms part of disclosed liquid compositions may have a weight ratio of omega-6 fatty acids: omega 9-fatty acids: omega-3 fatty acids of about 1: about 1.7 to about 2.3: about 5.7 to about 6.3. In other embodiments, the fatty acid composition has a weight ratio of omega-6 fatty acids: omega 9-fatty acids: omega-3 fatty acids of about 1: about 1.6 to about 2.4: about 5.6 to about 6.3. In other embodiments, the fatty acid composition has a weight ratio of omega-6 fatty acids: omega 9-fatty acids: omega-3 fatty acids of about 1: about 1.8 to about 2.2: about 5.8 to about 6.2. In other embodiments, the fatty acid composition has a weight ratio of omega-6 fatty acids: omega 9-fatty acids: omega-3 fatty acids of about 1: about 1.9 to about 2.1: about 5.9 to about 6.2. In other embodiments, the fatty acid composition has a weight ratio of omega-6 fatty acids: omega 9-fatty acids: omega-3 fatty acids of about 1: about 2.0 to about 2.2: about 6.0 to about 6.2. In other embodiments, the fatty acid composition has a weight ratio of omega-6 fatty acids: omega 9-fatty acids; omega-3 fatty acids of about 1: about 2.1 to about 2.2: about 6.1 to about 6.2. In other embodiments, the fatty acid composition has a weight ratio of omega-6 fatty acids: omega 9-fatty acids: omega-3 fatty acids of about 1: about 2.2: about 6.2. In other embodiments, the fatty acid composition has a weight ratio of omega-6 fatty acids: omega 9-fatty acids: omega-3 fatty acids of about 1: about 2.0: about 6.0. In other embodiments, the fatty acid composition has a weight ratio of omega-6 fatty acids: omega 9-fatty acids: omega-3 fatty acids of about 1: about 1.7 to about 2.3: about 3.0 to about 9.0, e.g., about 3.0, about 3.5, about 4.0, about 4.5, about 5.0, about 5.5, about 6.0, about 7.0, about 7.5, about 8.0, about 8.5, about 9.0.

For example, a contemplated fatty acid composition that forms part of disclosed liquid compositions may include the omega-3 fatty acids eicosapentaenoic acid, docosahexaenoic acid, and α-linoleic acid. For example, in some embodiments the weight ratio of eicosapentaenoic acid: docosahexaenoic acid: α-linoleic acid is about 1: about 1.8 to about 2.2: about 3.5 to about 4.1. In other embodiments, the weight ratio of eicosapentaenoic acid: docosahexaenoic acid: α-linoleic acid is about 1: about 1.8 to about 2.2: about 3.6 to about 4.0. In other embodiments, the weight ratio of eicosapentaenoic acid: docosahexaenoic acid: α-linoleic acid is about 1: about 1.9 to about 2.1: about 3.7 to about 3.9. In other embodiments, the weight ratio of eicosapentaenoic acid: docosahexaenoic acid: α-linoleic acid is about 1: about 2.0: about 3.8. In other embodiments, the fatty acid composition component may include, for example, about 200 to about 1500 mg eicosapentaenoic acid, e.g., about 200 to about 1000 mg eicosapentaenoic acid, e.g., about 200 to about 500 mg eicosapentaenoic acid, e.g., about 200 to about 300 mg eicosapentaenoic acid, e.g., about 250 to about 300 mg eicosapentaenoic acid, e.g., about 282 mg eicosapentaenoic acid; about 450 to about 3000 mg docosahexaenoic acid; e.g., about 450 to about 2000 mg docosahexaenoic acid; e.g., about 450 to about 1000 mg docosahexaenoic acid; e.g., about 450 to about 750 mg docosahexaenoic acid; e.g., about 500 to about 600 mg docosahexaenoic acid; e.g., about 550 to about 600 mg docosahexaenoic acid; e.g., about 582 mg docosahexaenoic acid; and about 1000 to about 3000 mg α-linoleic acid, e.g., about 1000 to about 2000 mg α-linoleic acid, e.g., about 1000 to about 1500 mg α-linoleic acid, e.g., about 1000 to about 1200 mg α-linoleic acid, e.g., about 1067 mg α-linoleic acid.

In some embodiments, a contemplated fatty acid composition that forms part of disclosed liquid compositions may further comprise vitamin D3 and/or vitamin E. For example, in other embodiments, the fatty acid composition further comprises vitamin E. In other embodiments, the fatty acid composition further comprises vitamin E. In other embodiments, the fatty acid composition further comprises vitamin D3 and vitamin E. In some embodiments, the fatty acid composition further comprises about 12.5 to about 37.5 mcg vitamin D3. e.g., about 15 to about 35 mcg vitamin D3, e.g., about 17.5 to about 32.5 mcg vitamin D3, e.g., about 20 to about 30 mcg vitamin D3, e.g., about 22.5 to about 27.5 mcg vitamin D3, e.g., about 25 mcg vitamin D3; and vitamin E, e.g., about 8 mg vitamin E.

In some embodiments, a disclosed liquid composition has a shelf life of 3 months or more when stored at 4° C. In other embodiments, the liquid composition has a shelf life of 4 weeks or more when stored at 20° C. In other embodiments, the shelf life of the emulsion forming liquid composition is determined by metagenomic analysis. In other embodiments, the shelf life of the emulsion forming liquid composition is determined by culture analysis. In other embodiments, the shelf life of the emulsion forming liquid composition is determined by metagenomic analysis and culture analysis.

A contemplated emulsion-forming liquid composition may have a pH of about 3.2 to about 3.6 at 20° C. In other embodiments, the shelf-stable, emulsion-forming liquid composition has a pH of about 3.3 to about 3.5, e.g., 3.3, 3.4, 3.5, at 20° C. In other embodiments, the shelf-stable, emulsion-forming liquid composition has a pH of about 3.3 to about 3.8, e.g., 3.3, 3.4, 3.5, 3.6, 3.7, 3.8 at 20° C.

For example, disclosed herein is an emulsion-forming liquid composition, comprising: a) an aqueous liquid composition having a pH of about 3.0 to about 3.9 as measured at 20° C., comprising at least two different amino acids each independently selected from the group consisting of tryptophan, 5-hydroxytryptophan, glycine, tyrosine, and phenylalanine; and b) a fatty acid composition comprising at least three omega-3 fatty acids; w % herein the aqueous liquid composition and the fatty acid composition form an amino acid/fatty acid buffer system allowing the creation of a fatty acid vesicle-based emulsion when the aqueous liquid composition and the fatty acid composition are shaken together.

In some embodiments, contemplated aqueous liquid composition portions of a disclosed liquid composition may include, for example, L-tryptophan and glycine. In some embodiments, the L-tryptophan and glycine are present in a weight ratio of about 1: about 6 to about 7.

In other embodiments, contemplated fatty acid composition portions of a disclosed liquid composition may include, for example, eicosapentaenoic acid, docosahexaenoic acid, and α-linoleic acid. For example, in some embodiments the weight ratio of eicosapentaenoic acid: docosahexaenoic acid: α-linoleic acid is about 1: about 1.8 to about 2.2: about 3.5 to about 4.1. In other embodiments, the weight ratio of eicosapentaenoic acid: docosahexaenoic acid; α-linoleic acid is about 1: about 1.8 to about 2.2: about 3.6 to about 4.0. In other embodiments, the weight ratio of eicosapentaenoic acid: docosahexaenoic acid: α-linoleic acid is about 1: about 1.9 to about 2.1: about 3.7 to about 3.9. In other embodiments, the weight ratio of eicosapentaenoic acid: docosahexaenoic acid: α-linoleic acid is about 1: about 2.0: about 3.8. In other embodiments, the fatty acid composition component may include, for example, about 200 to about 1500 mg eicosapentaenoic acid. e.g., about 200 to about 1000 mg eicosapentaenoic acid, e.g., about 200 to about 500 mg eicosapentaenoic acid, e.g., about 200 to about 300 mg eicosapentaenoic acid, e.g., about 250 to about 300 mg eicosapentaenoic acid, e.g., about 282 mg eicosapentaenoic acid; about 450 to about 3000 mg docosahexaenoic acid; e.g., about 450 to about 2000 mg docosahexaenoic acid; e.g., about 450 to about 1000 mg docosahexaenoic acid; e.g., about 450 to about 750 mg docosahexaenoic acid: e.g., about 500 to about 600 mg docosahexaenoic acid; e.g., about 550 to about 600 mg docosahexaenoic acid: e.g., about 582 mg docosahexaenoic acid; and about 1000 to about 3000 mg α-linoleic acid, e.g., about 1000 to about 2000 mg α-linoleic acid, e.g., about 1000 to about 1500 mg α-linoleic acid, e.g., about 1000 to about 1200 mg α-linoleic acid, e.g., about 1067 mg α-linoleic acid.

In some embodiments, the fatty acid composition further comprises vitamin D3 and/or vitamin E. For example, in other embodiments, the fatty acid composition further comprises vitamin E. In other embodiments, the fatty acid composition further comprises vitamin E. In other embodiments, the fatty acid composition further comprises vitamin D3 and vitamin E. In some embodiments, the fatty acid composition further comprises about 12.5 to about 37.5 mcg vitamin D3, e.g., about 15 to about 35 mcg vitamin D3, e.g., about 17.5 to about 32.5 mcg vitamin D3, e.g., about 20 to about 30 mcg vitamin D3, e.g., about 22.5 to about 27.5 mcg vitamin D3, e.g., about 25 mcg vitamin D3; and vitamin E, e.g., about 8 mg vitamin E.

A contemplated emulsion-forming liquid composition may further include magnesium, zinc, and/or a salt or hydrate thereof. For example, in some embodiments the emulsion-forming liquid composition further comprises magnesium (e.g., magnesium L-ascorbate dihydrate) and zinc (e.g., zinc citrate trihydrate.)

Also provided herein is a liquid beverage composition comprising L-tryptophan: glycine; one or more omega-6 fatty acids: one or more omega-9 fatty acids; and three or more omega-3 fatty acids: wherein the weight ratio of omega-6 fatty acids: omega-9 fatty acids; omega-3 fatty acids is about 1: about 1.7 to about 2.3: about 5.7 to about 6.3: and an effective amount of a food-compatible acidic agent; wherein the liquid beverage composition has a pH of about 3.2 to about 3.6 as measured at 20° C.

Exemplary omega-3 fatty acids may include, for example, docosahexaenoic acid, eicosapentaenoic acid and α-linoleic acid, where for example the weight ratio of eicosapentaenoic acid: docosahexaenoic acid: α-linoleic acid is about 1: about 1.8 to about 2.2: about 3.5 to about 4.1, about 1: about 1.8 to about 2.2: about 3.6 to about 4.0, about 1.9 to about 2.1: about 3.7 to about 3.9, or about 1: about 2.0: about 3.8). The liquid beverage composition may further include, for example, magnesium L-ascorbate dihydrate and zinc citrate trihydrate. The liquid beverage composition may also include, for example, vitamin D3 and vitamin E.

In some embodiments the food-compatible acidic agent is citric acid. Such a food-compatible acidic agent may be in amount such that the liquid or beverage has pH of about 3.2 to about 3.6, e.g., 3.2, 3.3, 3.5, 3.6, at 20° C.: or about 3.3 to about 3.5, e.g., 3.3, 3.4, 3.5, at 20° C.; or about 3.2 to about 3.8, e.g., 3.2, 3.3, 3.4, 3.5, 3.6, 3.7, 3.8, at 20° C. The citric acid may be present in the form of a fruit juice, as described above. For example, the citric acid is present in the form of lime juice and/or lemon juice. For example, the citric acid is present in the form of lime juice and lemon juice.

Also provided herein is a liquid beverage composition comprising flaxseed oil, algae oil, a plant-derived oil, L-tryptophan, and glycine wherein for example, the omega-6 fatty acids, omega-9 fatty acids and omega-3 fatty acids present in the flaxseed oil, algae oil, and plant-derived oil combined are present in a weight ratio of omega 6 fatty acids: omega 9 fatty acids: omega 3 fatty acids of about 1: about 1.7 to about 2.3: about 5.7 to about 6.3. For example, a liquid beverage composition is provided comprising: flaxseed oil, algae oil, a plant-derived oil, L-tryptophan, and glycine: wherein the eicosapentaenoic acid, docosahexaenoic acid, and α-linoleic acid present in the flaxseed oil and algae oil combined are present in a weight ratio of eicosapentaenoic acid: docosahexaenoic acid: α-linoleic acid of about 1: about 1.8 to about 2.2: about 3.5 to about 4. Such a composition may further comprise citric acid in an amount whereby the liquid beverage composition has a pH of about 3.2 to about 3.6 at 20° C., e.g., 3.2, 3.3, 3.4, 3.5, 3.6, at 20° C. In other embodiments, the liquid beverage composition further comprises citric acid in an amount whereby the liquid beverage composition has a pH of about 3.3 to about 3.5 at 20° C., e.g., 3.3, 3.4, 3.5, at 20° C. In other embodiments, the liquid beverage composition further comprises citric acid in an amount whereby the liquid beverage composition has a pH of about 3.3 to about 3.8, e.g., 3.3, 3.4, 3.5, 3.6, 3.7, 3.8, at 20° C.

In some embodiments, the L-tryptophan and glycine are present in the liquid beverage or liquid composition in a weight ratio of about 1: about 6 to about 7.

In some embodiments, the plant-derived oil is selected from the group consisting of coconut oil, avocado oil, olive oil, and mixtures thereof. In other embodiments, a plant-derived oil is coconut oil. In other embodiments, the plant-derived oil is avocado oil. In other embodiments, the plant-derived oil is olive oil. In other embodiments, a plant-derived oil is a mixture of olive oil and avocado oil. In other embodiments, a plant-derived oil is a mixture of olive oil and coconut oil. In other embodiments, a plant-derived oil is a mixture of avocado oil and coconut oil. In other embodiments, a plant-derived oil is a mixture of olive oil, avocado oil and coconut oil.

A fatty acid vesicle-forming liquid composition that may, for example, increase neurosystem uptake of amino acids present in the fatty acid vesicle-forming liquid composition when consumed by a subject, is also provided that comprises, per 60 ml of the liquid composition: 1-5 ml flaxseed oil; about 1-5 ml algae oil: about 1-5 ml of a plant-derived oil, wherein the plant-derived oil is selected from the group consisting of olive oil, avocado oil, coconut oil, and mixtures thereof: about 2000 to about 5000 mg glycine, e.g., about 2500 to about 3500 mg glycine, e.g., 3000 mg glycine; about 350 to about 700 mg tryptophan, e.g., about 400 to about 470 mg tryptophan, e.g., 450 mg L-tryptophan; and about 45 to about 57 ml, e.g., about 54 ml of a food-compatible aqueous solution with a pH of about 3.0 to about 3.9 at 20° C.: wherein when the liquid composition is shaken for e.g., 10 seconds or longer, (e.g., about 10, 20, 30, 40 seconds or more) the creation of fatty acid vesicles occurs; thereby effecting increased neurosystem uptake of the amino acids present in the liquid composition when consumed by the subject. The fatty-acid vesicle-forming liquid composition may further comprise, per 60 ml of the liquid composition about 25 mcg vitamin D3 (e.g., about 12.5 to about 37.5 mcg vitamin D3) and/or about 8 mg vitamin E; and optionally about 8 mg of zinc and/or about 326 (or e.g., 200-350) mg vitamin C and/or about 19 mg magnesium.

For example, a fatty acid vesicle-forming liquid composition that may, for example, increase neurosystem uptake of amino acids present in the fatty acid vesicle-forming liquid composition when consumed by a subject, is also provided that comprises, per 60 ml of the liquid composition: A fatty acid vesicle-forming liquid composition comprising, per 60 ml of the liquid composition: about 2 ml flaxseed oil; about 2 ml algae oil: about 2 ml of a plant-derived oil, wherein the plant-derived oil is selected from the group consisting of olive oil, avocado oil, coconut oil, and mixtures thereof: about 400 mg L-phenylalanine; about 200 mg L-tryptophan; and about 54 ml of a food-compatible aqueous solution with a pH of about 3.0 to about 3.9 at 20° C.; wherein when the liquid composition is shaken for e.g., 10 seconds or longer, (e.g., about 10, 20, 30, 40 seconds or more) the creation of fatty acid vesicles occurs; thereby effecting increased neurosystem uptake of the amino acids present in the liquid composition when consumed by the subject.

The fatty-acid vesicle-forming liquid composition may further comprise, per 60 ml of the liquid composition about 25 mcg vitamin D3 (e.g., about 12.5 to about 37.5 mcg vitamin D3) and/or about 8 mg vitamin E: and optionally about 8 mg of zinc and/or about 326 (or e.g., 200-350) mg vitamin C and/or about 19 mg magnesium.

In some embodiments, the plant-derived oil is olive oil. In other embodiments, the plant-derived oil is avocado oil. In other embodiments, the plant-derived oil is coconut oil. In other embodiments, the plant-derived oil is a mixture of olive oil and avocado oil. In other embodiments, the plant-derived oil is a mixture of olive oil and coconut oil. In other embodiments, the plant-derived oil is a mixture of avocado oil and coconut oil. In other embodiments, the plant-derived oil is a mixture of olive oil, avocado oil and coconut oil.

In some embodiments, the food-compatible aqueous solution comprises, per 60 ml of the liquid composition: about 40 ml coconut water: about 12 ml lime juice: and about 2 ml lemon juice.

The algae oil present in the fatty acid vesicle-forming liquid composition may be, for example, Schizochytrium sp. oil.

Also provided herein, for example, is method of improving sleep, well-being and/or mood in a human subject in need thereof, comprising administering to the human subject an effective amount of a composition disclosed herein, for example, a disclosed emulsion-forming liquid composition, a disclosed liquid beverage composition, or a disclosed fatty acid vesicle-forming liquid composition.

Further disclosed herein, for example, is a method of treatment or prophylaxis of depression in a human subject in need thereof, comprising administering to the human subject an effective amount of a composition disclosed herein, for example, a disclosed, emulsion-forming liquid composition, a disclosed liquid beverage composition, or a disclosed fatty acid vesicle-forming liquid composition.

### Examples

The features and other details of the disclosure will be more readily understood by reference to the following examples, which are included merely for purposes of illustration of certain aspects and embodiments of the present disclosure, and are not intended to limit the invention in any way. No animals were used in the following examples.

### Example 1: Composition

A liquid composition of the present disclosure was prepared according to Table 1.

TABLE 1

| Raw material | Type | Amount | Unit | Active ingredient | Amount | Unit |
|---|---|---|---|---|---|---|
| Coconut water | Liquid | 40 | ml | | | |
| Lime juice | Liquid | 12 | ml | | | |
| Lemon juice | Liquid | 2 | ml | Citric acid | | |
| Vitamin D3 oil | Oil | 0.05 | ml | Vitamin D3 | 25 | mcg |
| Flaxseed oil | Oil | 2 | ml | Alpha-Linoleic acid | 1067 | mg |
| | | | | Omega-6 | 260 | mg |
| | | | | Omega-9 | 360 | mg |
| | | | | Vitamin E | 1 | mg |
| Algae oil | Oil | 2 | ml | Eicosapentaenoic acid | 282 | mg |
| | | | | Docosahexaenoic acid | 564 | mg |
| | | | | Other Omega-3 | | |
| | | | | fatty acids | 188 | mg |
| | | | | Omega-6 | 20 | mg |
| | | | | Omega-9 | 220 | mg |
| | | | | Vitamin E | 4 | mg |
| Coconut oil | Oil | 2 | ml | Omega-6 | 60 | mg |
| | | | | Omega-9 | 150 | mg |
| Vitamin E oil | Oil | 0.05 | ml | Vitamin E | 8 | mg |
| Magnesium L-ascorbate | | | | | | |
| Dihydrate | Solid | 345 | mg | Vitamin C | 326 | mg |
| | | | | Magnesium | 19 | mg |
| Zinc citrate trihydrate | Solid | 25 | mg | Zinc | 8 | mg |
| Glycine | Solid | 3000 | mg | Glycine | 3000 | mg |
| Tryptophan | Solid | 450 | mg | Tryptophan | 450 | mg |
| pH Total | 3.2-3.6 | | | | | |
| High pressure processing | 2 cycles time 5 min | | | | | |
| Shelf-life | >3 months | | 4° C. | | | |
| Shelf-life | >1 month | | 20° C. | | | |
| Omega −3:−6:−9 ratio | | | | | | Ratio |
| | | | | ALA | 1067 | |
| | | | | DHA | 564 | |
| | | | | EPA | 282 | |
| | | | | Other omega-3 | 188 | |
| | | | | Omega-3 | 2101 | 6 |
| | | | | Omega-6 | 340 | 1 |
| | | | | Omega-9 | 730 | 2 |

### Example 2: Chemical and Microbial Stability and Shelf Life

The chemical stability of a disclosed liquid composition stored at 6° C. or 22° C. was evaluated at 2 months and three months. The results are shown in Table 2. The microbial stability and shelf life was also evaluated. A liquid composition having a pH of about 3.7 was subjected to high pressure processing at 6000 bar for two cycles of 5 minutes. The treatment provided a shelf-life of at least 14 weeks at 6° C. or 22° C. as confirmed by metagenome analysis, culture analysis and colony count, with no significant changes in levels of e.g., lactic acid bacteria. Enterobacteriaceae, E. coli, yeast, mold, listeria and salmonella, and no significant changes in pH.

TABLE 2

| | | Label | 2 Months | 3 Months |
|---|---|---|---|---|
| Vitamin C | mg/60 mL | 280 | 326 | 245 |
| Vitamin D | mcg/60 mL | 25 | 25 | 26* |
| Vitamin E | mg/60 mL | 8 | 5.6 | 2.2 |
| Magnesium | mg/60 mL | 20 | 15 | 27.8 |
| Zinc | mg/60 mL | 8 | 6 | 5.7 |
| Sodium | mg/60 mL | 0 | 1.23 | 0.6 |
| Glycine | mg/60 mL | 3000 | 3319 | 3303 |
| Medium chain | mg/60 mL | 1500 | 2209 | 2407* |
| triglycerides | | | | |
| alpha linoleic acid | mg/60 mL | 1100 | 1026 | 1025* |
| Omega 9 fatty acids | mg/60 mL | 730 | 586 | 580* |
| docosapenataenoic acid | mg/60 mL | 560 | 632 | 587* |
| L-Tryptophan | mg/60 mL | 450 | 420 | 426 |
| eicosapentaenoic acid | mg/60 mL | 280 | 319 | 297* |

*Adjusted for slightly lower oil volume in 3 months preparation (60 ml in 2 months sample vs 55 ml in 3 months sample)

### Example 3: Pharmacological Studies

Using the composition according to Table 1, the effects on sleep quality, mood and well-being in a subject population were investigated as follows.

### Study Design

Changes in sleep quality were measured by biosensory data metrics using an Oura® ring fitted to each subject. Examples of these metric were, e.g., time to reach first deep sleep phase, number of wakeups during the night, and overall sleep duration. Sleep quality, mood and well-being was also measured by subjective endpoints, for example, a self-assessment questionnaire compiled from Pittsburgh Sleep Quality Index (PSQI) and Hamilton Depression Scale 17 (HAMD 17) self-assessment questionnaires.

The subjects were 35-55 years old, not using SSRI's or any sleeping medication, and not using any other nutritional supplements or herbal products to improve sleep. The nutrition preferences for each individual subject were noted (e.g., no limitations, vegan diet, or vegetarian diet).

Overall, 80 individuals (40 male, 40 female) were first followed over seven days, without any consumption of the liquid composition, to establish a baseline. Biosensory data by Oura ring and data from the compiled PSQI and HAMD 17 self-assessment questionnaire were collected to establish the baseline. On day 8, subjects were randomly assigned to four groups of 25 subjects each. Numerical balance for gender differences, age and diet preference within each group was ensured. Subjects were not given any further information on ingredients or composition specifics. Group A (control group) received 60 ml of fruit juice only; Group B received a liquid composition without glycine and tryptophan; Group C received first glycine and tryptophan as a solid powder, and one hour later a liquid composition without glycine and tryptophan; and Group D received the full, complete liquid composition. Groups B. C and D were advised to shake their respective compositions strongly before consumption. All groups were followed over a period of 7 days collection. Biosensory data was measured by Oura ring. Subjects completed a compiled PSQI and HAMD 17 self-assessment questionnaire each morning.

### Results

a) Comparison within each study group vs. own individual baseline:
   Group A: No meaningful changes in sleep, mood and wellbeing over time on any metrics.
   Group B: No significant improvement. Trend in mood and well-being improvement in subjects on vegan diet as measured by the self-assessment questionnaire (e.g., a happiness score increase by 1-2 scores).
   Group C: No significant improvement. Trend in mood and well-being improvement in subjects on vegan diet as measured by the elf-assessment questionnaire (e.g., a happiness score increase by 1-2 scores). Nine subjects experienced mild GI discomfort after taking the amino acid powders.
   Group D: Significant improvement in (i) biosensory data measured by Oura ring (longer overall sleep duration (+20%-+35%), longer deep sleep phase (+45%-+95%) and (ii) sleep/mood/well-being quality measured by subjective endpoints: “Falling asleep faster” in 25/25 subjects; “higher sleep quality” in 24/25 subjects; “higher sleep quantity” in 23/25 participants; “higher anxiety reduction” in 13/25 subjects: “feeling more happy in the morning” (improvement by 3 scores) in 18/25 subjects; and “feeling less depressed/moody in the morning” (improvement by at least 2 scores) in 16/25 subjects. Two subjects experienced mild GI discomfort.
b) Comparison between study groups (comparison of treatment week (2^{nd} week)):

No significant difference was observed between Group A. B. C. A significant improvement was observed for Group D over Groups A. B, and C on several measurements:
1) longer overall sleep duration (+20%-+35%);
2) longer deep sleep phase (+45%-+95%); and
3) sleep, mood and well-being as measured by subjective endpoints (“Falling asleep faster” in 25/25 subjects; “higher sleep quality” 24/25 participants; “higher sleep quantity” in 23/25 subjects; anxiety reduction in 13/25 subjects; “feeling happier in the morning” (improvement by 3 scores) in 18/25 subjects; “feeling less depressed/moody in the morning” (improvement by at least 2 scores) in 16/25 subjects. There were no reports of any discomfort and side effects in Group 4 over Groups 2 and 3.

### Example 4: Pharmacological Studies

The effects of a disclosed composition in drink form on mood, sleep quality, and energy level in a subject population of 22 subjects were investigated as follows.

On day 1, to establish baseline metrics, subjects did not receive a drink formula and completed a self-assessment questionnaire related to mood, sleep and well-being 30 minutes after waking up in the morning. On day 2, subjects were randomized into four groups of 7, 5, 5 and 5 subjects, and each group consumed a different drink formula 15-20 minutes before bedtime. Group D (7 subjects) consumed a drink formula containing all ingredients, Group NO (5 subjects) received a drink formula with no oil ingredients, Group NM (5 subjects) received a drink formula with no micronutrient ingredients, and Group P (5 subjects) received a placebo drink. On day 3, all subjects again completed the same self-assessment questionnaire 30 minutes after waking up in the morning. The drink formulae per treatment group are shown in Table 3.

TABLE 3
Drink formulae per treatment group

| | Group D | Group NO | Group NM | Group P |
|---|---|---|---|---|
| algae oil | + | − | + | − |
| flaxseed oil | + | − | + | − |
| MCT oil | + | − | + | − |
| wheat germ oil | − | − | − | + |
| sunflower oil | − | − | − | + |
| glycine | + | + | − | − |
| tryptophan | + | + | − | − |
| vitamin C | + | + | − | − |
| vitamin E | + | + | − | − |
| vitamine D3 | + | + | − | − |
| magnesium | + | + | − | − |
| lemon juice | + | + | + | + |
| # of subjects | 7 | 5 | 5 | 5 |

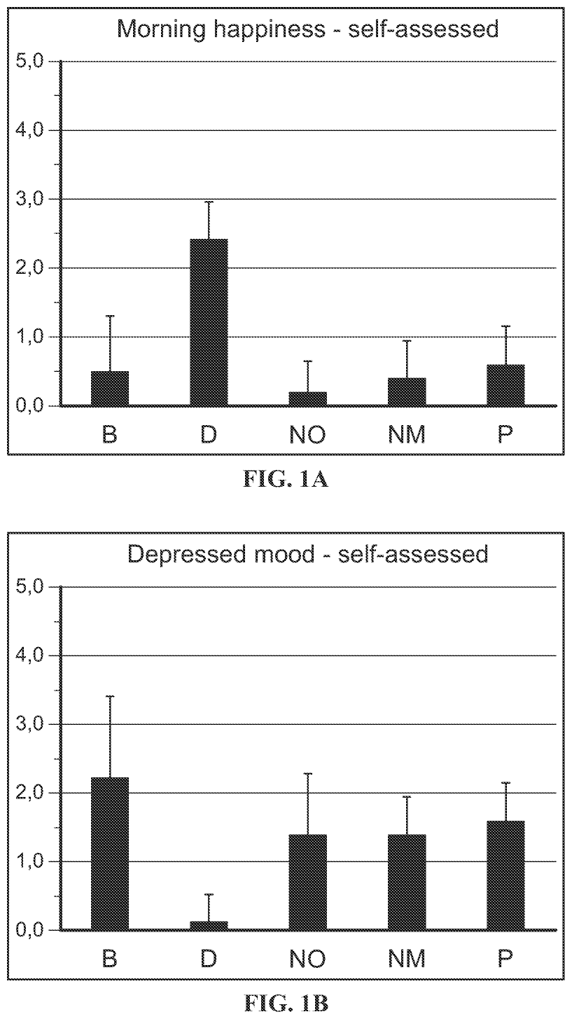

As shown in FIG. 1A. Group D reported significant improvement over baseline in feeling more happy in the morning (self-reported in a scale from 0-5) compared to all other groups. No apparent effect was observed in groups drinking formula without omega-3 oils (NO), without micronutrients (NM) or placebo (P).

As shown in FIG. 1B, Group D reported significant improvement in feeling depressed in the morning (self-reported in a scale from 0-5) compared to all other groups, with each participant in Group D reporting substantial reduction of depressed feelings. No apparent effect was observed in groups drinking formula without omega-3 oils (NO), without micro-nutrients (NM) or placebo (P).

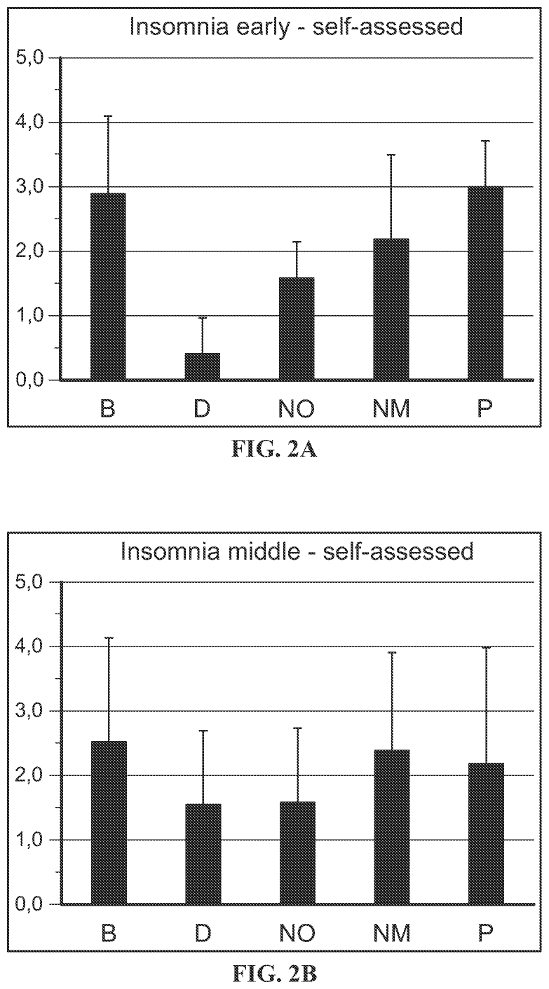

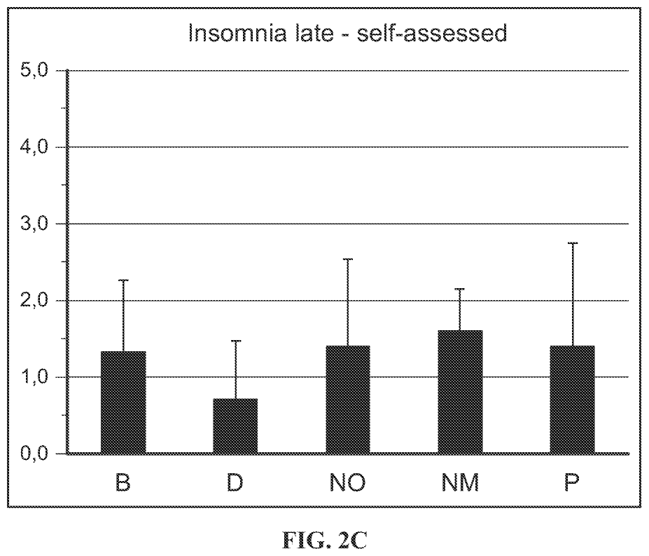

As shown in FIG. 2A, Group D reported significant improvement in falling asleep faster (insomnia early) (self-reported in a scale from 0-5) compared to all other groups. Time needed to fall asleep was accelerated on average by more than 30 min. Some improvement over baseline was observed in the group drinking formula without oil but with micro-nutrients (“NO”). No apparent effect was observed in groups drinking formula without micro-nutrients (NM) or placebo. As shown in FIG. 2B, no apparent improvement over baseline on disruption of sleep during the night (insomnia middle) was observed in any of the groups. As shown in FIG. 2C, no apparent effect on disruption of sleep early morning hours (insomnia late) was observed in any of the groups.

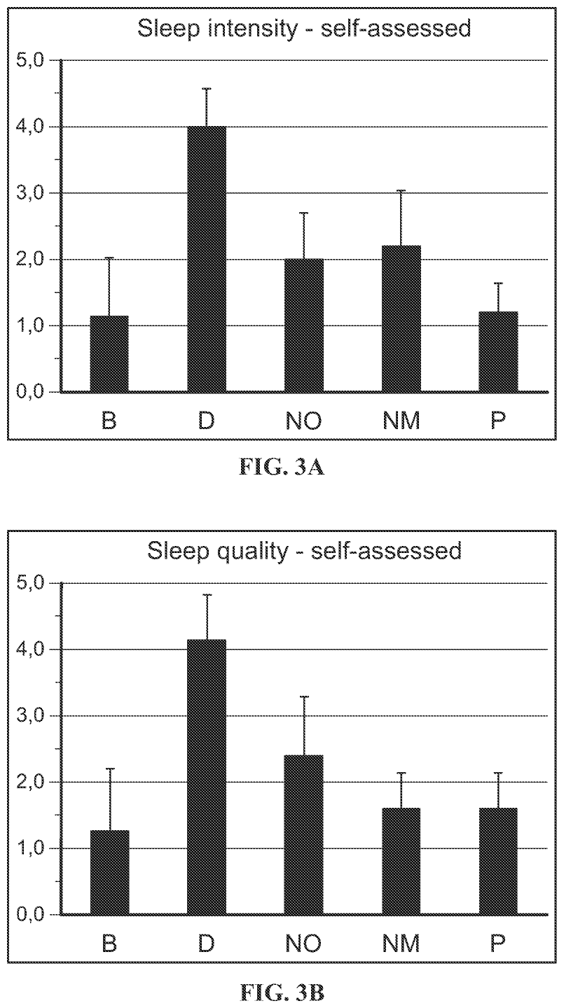

As shown in FIG. 3A, Group D reported significant improvement over baseline in sleep intensity (sleep depth based on self-reporting in a scale from 0-5) compared to all other groups. Some improvement over baseline was observed for groups drinking formula without omega-3 oils (NO) and without micro-nutrients (NM). No apparent effect was observed for the group drinking placebo (P).

As shown in FIG. 3B, Group D reported significant improvement over baseline in sleep quality (self-reported in a scale from 0-5) up to the highest levels of perceived sleep quality. Some improvement over baseline was observed for the group drinking formula without omega-3 oil (NO). No apparent effect was observed for the groups drinking formula without micro-nutrients (NM) or placebo (P).

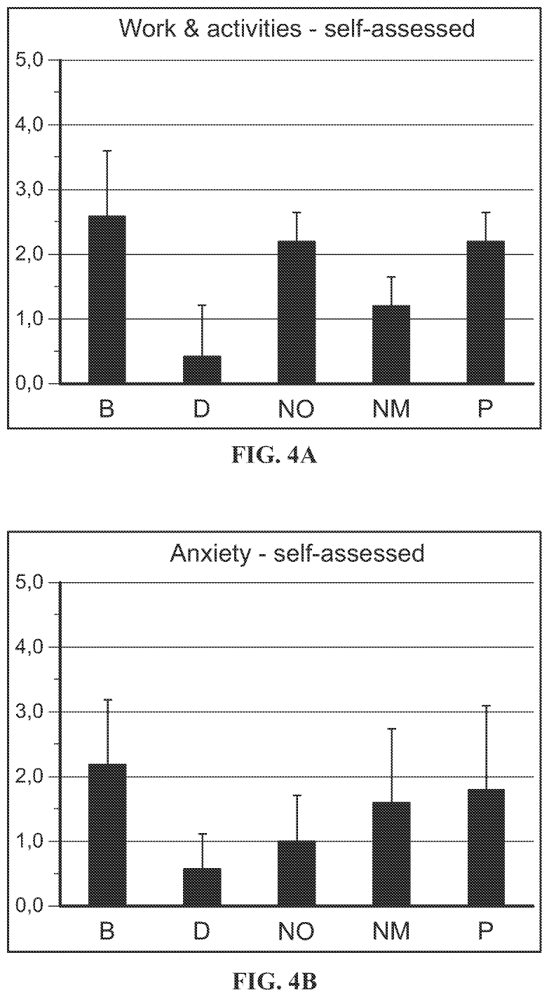

As shown in FIG. 4A, Group D reported significant improvement in perceived energy level for work and activities the next day (self-reported in a scale from 0-5) compared to the placebo group (P) and the group drinking formula without oil (NO). Some improvement was observed in the group drinking formula without micronutrients (NM).

As shown in FIG. 4B, Group D reported significant improvement anxiety (self-reported in a scale from 0-5) compared to the placebo group (P), the group drinking formula without oil (NO) and baseline (B). In Group D, all participants reporting substantial anxieties (rating 3-5) showed reduction by 1 or 2 scores. Some improvement was observed in the group drinking formula without omega-3 oils (NM). No apparent effect was observed in the placebo group (P) or the group drinking formula without micronutrients (NM).

### Example 5: Pharmacological Studies

The effects of a disclosed composition in drink form on mood and sleep quality in a subject population of 114 male and female subjects between 25-60 years old, over a period of 6 weeks, were investigated as follows.

On day 1, to establish baseline metrics, subjects and completed a self-assessment questionnaire related to sleep quality, followed by consumed the drink formula before bedtime. On day 2, all subjects again completed the same self-assessment questionnaire 30 minutes after waking up in the morning. All subjects were then free to decide the day on which to consume one additional drink formula before bedtime, followed by completion of the self-assessment questionnaire 30 minutes after waking up the next morning. Seven weeks after test initiation, all subjects who completed the self-assessment questionnaire were included for data analysis on sleep quality, onset of sleep (insomnia early) and self-assessment of “feeling in the morning.”

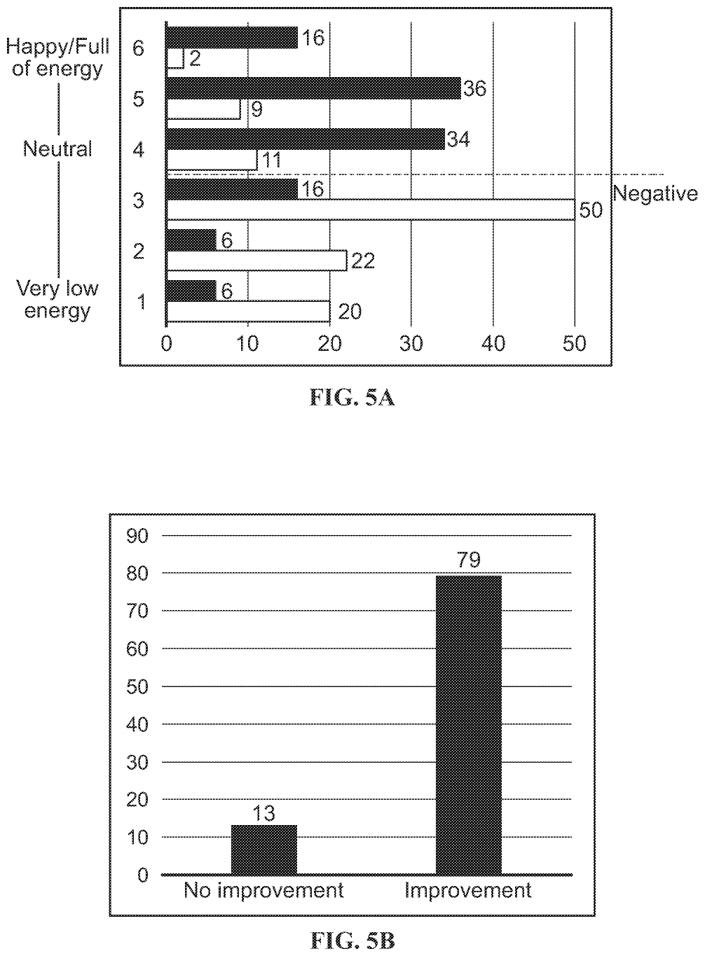

FIG. 5A depicts results of “feeling in the morning” self-assessments of subjects who consumed the drink formula (black bars) compared to baseline (no consumption of drink, white bars). Scores were recorded from a range of 1 (very low energy) to 6 (happy/full of energy). After using the drink formula, 75% (86) of subjects assessed their morning feelings as neutral or positive. In the initial baseline assessment, 19% (22) of subjects assessed their morning feelings as neutral or positive, while 81% (92) of subjects assessed their morning feelings as negative. Of that 81% (92) of subjects who assessed their morning feelings as negative, 86% (79) of subjects showed an improvement in the self-assessment questionnaire after using the drink formula, as shown in FIG. 5B.

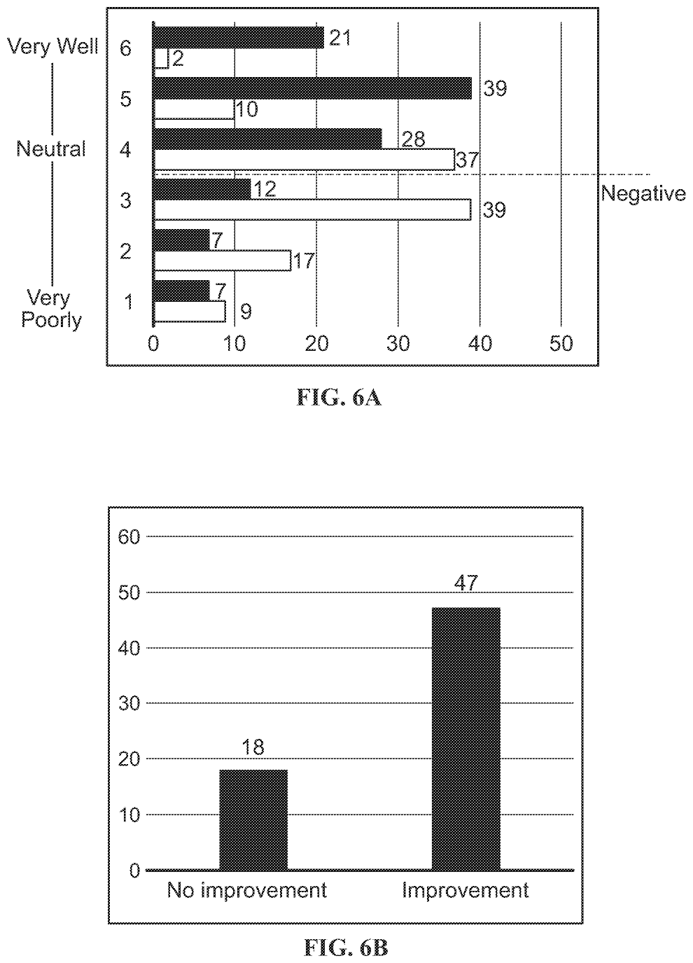

FIG. 6A depicts results of sleep quality self-assessments of subjects who consumed the drink formula (black bars) compared to baseline (no consumption of drink, white bars). Scores were recorded from a range of 1 (very poorly) to 6 (very well). After using the drink formula, 77% (88) of subjects assessed their sleep quality as neutral or positive. In the initial baseline assessment, 43% (49) of subjects assessed their sleep quality as neutral (32%, 37) or positive (11%, 12), while 57% (65) of subjects assessed their sleep quality. Of that 57% (65) of subjects who assessed their sleep quality, 72% (47%) of subjects showed an improvement in the self-assessment questionnaire after using the drink formula, as shown in FIG. 6B.

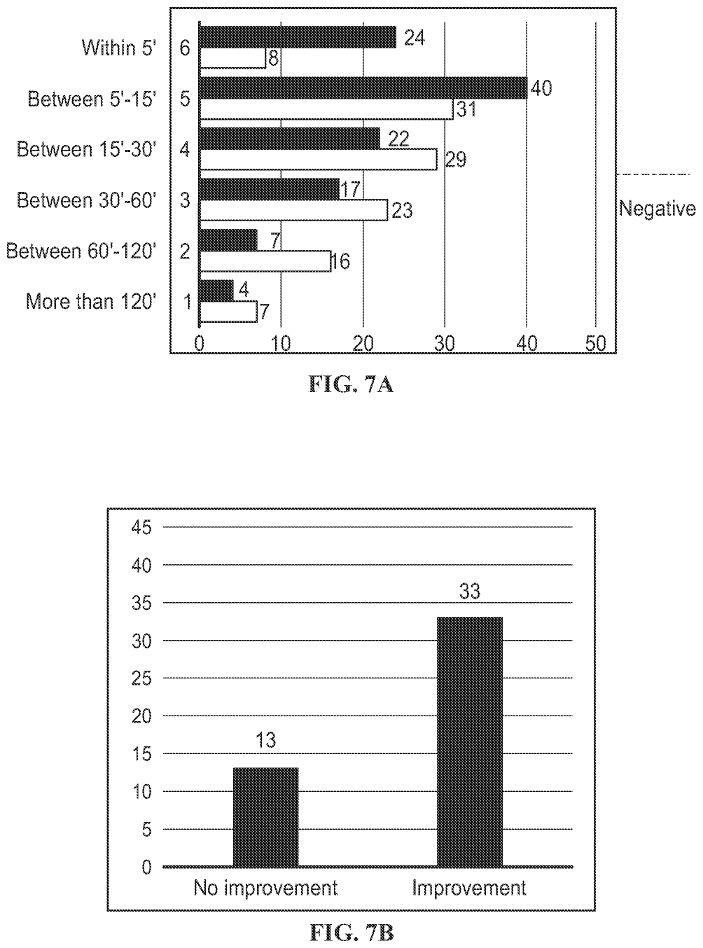

FIG. 7A depicts results of “time needed to fall asleep” self-assessments of subjects who consumed the drink formula (black bars) compared to baseline (no consumption of drink, white bars). Scores were recorded from a range of 1 (more than 120 min) to 6 (less than 5 min.) After using the drink formula, 75% (88) of subjects reported falling asleep within 30 minutes. In the initial baseline assessment, 60% (68) reported falling asleep within 30 minutes, while 40% (46) of subjects reported falling asleep after 30 minutes or later (delayed sleep onset). Of that 40% (46) of subjects who reported delayed sleep onset, 72% (33%) of subjects showed an improvement in the self-assessment questionnaire after using the drink formula, as shown in FIG. 7B.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. An emulsion-forming liquid composition, comprising:
an aqueous liquid composition having a pH of about 3.0 to about 3.9 as measured at 20° C., comprising at least two different amino acids each independently selected from the group consisting of tryptophan, 5-hydroxytryptophan, glycine, tyrosine, and phenylalanine; and
a fatty acid composition comprising at least one omega-9 fatty acid, at least one omega-6 fatty acid; and at least three omega-3 fatty acids;
wherein the aqueous liquid composition and the fatty acid composition form an amino acid/fatty acid buffer system allowing the creation of a fatty acid vesicle-based emulsion when the aqueous liquid composition and the fatty acid composition are shaken together.

2. The emulsion-forming liquid composition of claim 1, wherein the aqueous liquid composition and the fatty acid composition are contained within one container.

3. The emulsion-forming liquid composition of claim 1, wherein the aqueous liquid composition and the fatty acid composition are contained within separate containers before the emulsion-forming liquid composition is shaken.

4. The emulsion-forming liquid composition of any one of claims 1-3, wherein when consumed in emulsion form by a subject, provides increased neurosystem uptake of amino acids in the subject.

5. The emulsion-forming liquid composition of any one of claims 1-4, wherein the aqueous liquid composition further comprises a fruit juice.

6. The emulsion-forming liquid composition of claim 5, wherein the fruit juice comprises lemon and/or lime juice.

7. The emulsion-forming liquid composition of any one of claims 1-6, wherein the aqueous liquid composition further comprises coconut water.

8. The emulsion-forming liquid composition of any one of claims 1-7, wherein the amino acids present in the aqueous liquid composition may each independently be an L-isomer or a mixture of D- and L-isomers.

9. The emulsion-forming liquid composition of any one of claims 1-8, wherein the aqueous liquid composition comprises L-tryptophan and glycine.

10. The emulsion-forming liquid composition of any one of claims 1-9, further comprising magnesium, zinc, and/or a salt or hydrate thereof.

11. The emulsion-forming liquid composition of any one of claims 1-10, further comprising magnesium L-ascorbate dihydrate and zinc citrate trihydrate.

12. The emulsion-forming liquid composition of any one of claims 1-11, wherein the fatty acid composition comprises eicosapentaenoic acid, docosahexaenoic acid, and α-linoleic acid.

13. The emulsion-forming liquid composition of claim 12, wherein the weight ratio of eicosapentaenoic acid: docosahexaenoic acid: α-linoleic acid is about 1: about 1.8 to about 2.2: about 3.5 to about 4.1.

14. The emulsion-forming liquid composition of any one of claims 1-13, wherein the fatty acid composition has a weight ratio of omega-6 fatty acids: omega 9-fatty acids: omega-3 fatty acids of about 1: about 1.7 to about 2.3: about 5.7 to about 6.3.

15. The emulsion-forming liquid composition of any one of claims 1-14, wherein the fatty acid composition comprises flaxseed oil, algae oil, and a plant oil selected from the group consisting of coconut oil, avocado oil, olive oil, and mixtures thereof.

16. The emulsion-forming liquid composition of any one of claims 1-15, wherein the fatty acid composition further comprises vitamin D3 and/or vitamin E.

17. The emulsion-forming liquid composition of any one of claims 1-16, wherein the liquid composition has a shelf life of 3 months or more when stored at 4° C. as determined by metagenomic analysis and/or culture analysis.

18. The emulsion-forming liquid composition of any one of claims 1-17, wherein the liquid composition has a shelf life of 4 weeks or more when stored at 20° C. as determined by metagenomic analysis and/or culture analysis.

19. The emulsion-forming liquid composition of any one of claims 1-18, wherein the shelf-stable, emulsion-forming liquid composition has a pH of about 3.2 to about 3.6 at 20° C.

20. An emulsion-forming liquid composition, comprising:
an aqueous liquid composition having a pH of about 3.0 to about 3.9 as measured at 20° C., comprising at least two different amino acids each independently selected from the group consisting of tryptophan, glycine, tyrosine, and phenylalanine; and
a fatty acid composition comprising at least three omega-3 fatty acids;
wherein the aqueous liquid composition and the fatty acid composition form an amino acid/fatty acid buffer system allowing the creation of a fatty acid vesicle-based emulsion when the aqueous liquid composition and the fatty acid composition are shaken together.

21. The emulsion-forming liquid composition of claim 20, wherein the aqueous liquid composition comprises L-tryptophan and glycine.

22. The liquid beverage composition of claim 21, wherein the L-tryptophan and glycine are present in a weight ratio of about 1: about 6 to about 7.

23. The emulsion-forming liquid composition of any one of claims 20-23, wherein the fatty acid composition comprises eicosapentaenoic acid, docosahexaenoic acid, and α-linoleic acid.

24. The emulsion-forming liquid composition of claim 23, wherein the weight ratio of eicosapentaenoic acid: docosahexaenoic acid: α-linoleic acid is about 1: about 1.8 to about 2.2: about 3.5 to about 4.1.

25. A liquid beverage composition comprising:
one or more omega-6 fatty acids;
one or more omega-9 fatty acids;
three or more omega-3 fatty acids;
wherein the weight ratio of omega-6 fatty acids: omega-9 fatty acids: omega-3 fatty acids is about 1: about 1.7 to about 2.3: about 5.7 to about 6.3:
L-tryptophan;
glycine; and
an effective amount of a food-compatible acidic agent; wherein the liquid beverage composition has a pH of about 3.2 to about 3.6 as measured at 20° C.

26. The liquid beverage composition of claim 25, wherein the food-compatible acidic agent is citric acid.

27. The liquid beverage composition of claim 26, wherein the citric acid is present in the form of a combination of lime and lemon juice.

28. The liquid beverage composition of any one of claims 25-27, further comprising vitamin D and/or vitamin E.

29. The liquid beverage composition of any one of claims 25-28, wherein the three or more omega-3 fatty acids comprises docosahexaenoic acid, eicosapentaenoic acid and α-linoleic acid.

30. The liquid beverage composition of any one of claims 25-29, further comprising magnesium L-ascorbate and zinc citrate.

31. A liquid beverage composition comprising:
flaxseed oil, algae oil, a plant-derived oil, L-tryptophan, and glycine;
wherein the omega-6 fatty acids, omega-9 fatty acids and omega-3 fatty acids present in the flaxseed oil, algae oil, and plant-derived oil combined are present in a weight ratio of omega 6 fatty acids: omega 9 fatty acids: omega 3 fatty acids of about 1:
about 1.7 to about 2.3: about 5.7 to about 6.3.

32. A liquid beverage composition comprising:
flaxseed oil, algae oil, plant-derived oil, L-tryptophan, and glycine;
wherein the eicosapentaenoic acid, docosahexaenoic acid, and α-linoleic acid present in the flaxseed oil, plant-derived oil, and algae oil combined are present in a weight ratio of eicosapentaenoic acid: docosahexaenoic acid: α-linoleic acid of about 1: about 1.8 to about 2.2: about 3.5 to about 4.1.

33. The liquid beverage composition of claim 32, wherein the L-tryptophan and glycine are present in a weight ratio of about 1: about 6 to about 7.

34. The liquid beverage composition of claim 32 or 33, further comprising citric acid in an amount whereby the liquid beverage composition has a pH of about 3.2 to about 3.6 at 20° C.

35. The liquid beverage composition of any one of claims 32-34, wherein the plant-derived oil is selected from the group consisting of coconut oil, avocado oil, olive oil, and mixtures thereof.

36. The liquid beverage composition of any one of claims 32-35, wherein the plant-derived oil is coconut oil.

37. A fatty acid vesicle-forming liquid composition comprising, per 60 ml of the liquid composition:
about 1-5 ml flaxseed oil;
about 1-5 ml algae oil;
about 1-5 ml of a plant-derived oil, wherein the plant-derived oil is selected from the group consisting of olive oil, avocado oil, coconut oil, and mixtures thereof:
about 2000 to about 5000 mg glycine;
about 350 to about 700 mg L-tryptophan; and
about 45-57 ml of a food-compatible aqueous solution with a pH of about 3.0 to about 3.9 at 20° C.;
wherein when the liquid composition is shaken for 10 seconds or longer, the creation of fatty acid vesicles occurs.

38. A fatty acid vesicle-forming liquid composition comprising, per 60 ml of the liquid composition:
about 2 ml flaxseed oil:
about 2 ml algae oil:
about 2 ml of a plant-derived oil, wherein the plant-derived oil is selected from the group consisting of olive oil, avocado oil, coconut oil, and mixtures thereof, about 3000 mg glycine;
about 450 mg L-tryptophan; and
about 54 ml of a food-compatible aqueous solution with a pH of about 3.0 to about 3.9 at 20° C.;
wherein when the liquid composition is shaken for 10 seconds or longer, the creation of fatty acid vesicles occurs.

39. The fatty acid vesicle-forming liquid composition of claim 38, further comprising, per 60 ml of the liquid composition:
about 25 mcg vitamin D3 and/or about 8 mg vitamin E; and
optionally about 8 mg zinc and/or about 326 mg vitamin C and/or about 19 mg magnesium.

40. The fatty acid vesicle-forming liquid composition of claim 38 or 39, wherein the food-compatible aqueous solution comprises, per 60 ml of the liquid composition:
about 40 ml coconut water;
about 12 ml lime juice; and
about 2 ml lemon juice.

41. The fatty acid vesicle-forming liquid composition of any one of claims 38-40, wherein the algae oil is Schizochytrium sp. oil.

42. A method of improving sleep, well-being and/or mood in a human subject in need thereof, comprising administering to the human subject an effective amount of a composition of any one of claims 1-41.

43. A method of treatment or prophylaxis of depression in a human subject in need thereof, comprising administering to the human subject an effective amount of a composition of any one of claims 1-41.
